(19) United States
(12) Patent Application Publication
(10) Pub. No.: US 2023/0317403 A1
(43) Pub. Date: Oct. 5, 2023

(54) PARTICLE BEAM IRRADIATION APPARATUS

(71) Applicant: SUMITOMO HEAVY INDUSTRIES, LTD., Tokyo (JP)

(72) Inventors: Takuya FUKUURA, Niihama-shi (JP); Masaru NAKAKITA, Niihama-shi (JP)

(21) Appl. No.: 18/188,458

(22) Filed: Mar. 23, 2023

(30) Foreign Application Priority Data
Mar. 29, 2022 (JP) 2022-053865

(51) Int. Cl. H01J 37/09; B23B 1/00; A61N 5/10

(52) U.S. Cl. CPC H01J 37/09; B23B 1/00; A61N 5/1045; H01J 2237/0473; H01J 2237/002; A61N 2005/1094; A61N 2005/1087

(57) ABSTRACT

A particle beam irradiation apparatus includes: an accelerator accelerating a particle so as to generate a particle beam; an irradiation unit irradiating an irradiation target with the particle beam; a transport path provided between the accelerator and the irradiation unit and provided so as to be capable of transporting the particle beam; and a collimator device having a first shielding member provided in the transport path, shielding the particle beam, and having a first opening allowing the particle beam to pass in an advancing direction of the particle beam and a second shielding member provided in the transport path, shielding the particle beam, and having a second opening allowing the particle beam to pass in the advancing direction of the particle beam, in which the second shielding member is spaced from the first shielding member to a downstream side in the advancing direction of the particle beam.

### CROSS-REFERENCE TO RELATED APPLICATIONS

This application claims priority to Japanese Patent Application No. 2022-053865, filed on Mar. 29, 2022, which is incorporated by reference herein in its entirety.

### BACKGROUND

### Technical Field

Certain embodiments of the present invention relate to a particle beam irradiation apparatus.

### Description of Related Art

Particle beam irradiation apparatuses are disclosed in the related art. A particle beam irradiation apparatus includes an accelerator accelerating a particle so as to generate a particle beam, an irradiation device performing irradiation with the particle beam generated by the accelerator, and a transport path for particle beam transport from the accelerator to the irradiation device. In this particle beam irradiation apparatus, a collimator collimates the particle beam emitted from the irradiation unit.

### SUMMARY

According to an embodiment of the present invention, there is provided a particle beam irradiation apparatus including: an accelerator accelerating a particle so as to generate a particle beam; an irradiation unit irradiating an irradiation target with the particle beam; a transport path provided between the accelerator and the irradiation unit and provided so as to be capable of transporting the particle beam; and a collimator device having at least a first shielding member and a second shielding member provided in the transport path, shielding the particle beam, and respectively having openings allowing the particle beam to pass in an advancing direction of the particle beam, in which the second shielding member is spaced from the first shielding member to a downstream side in the advancing direction of the particle beam, and the opening of the second shielding member is visible on the downstream side in a case where the opening of the first shielding member is viewed from an upstream side in the advancing direction of the particle beam.

A collimator device according to an embodiment of the present invention collimates a particle beam and includes: at least a first shielding member and a second shielding member shielding the particle beam and respectively having openings allowing the particle beam to pass in an advancing direction of the particle beam; and a support member supporting the first shielding member and the second shielding member, in which the second shielding member is spaced from the first shielding member to a downstream side in the advancing direction of the particle beam, and the opening of the second shielding member is visible on the downstream side in a case where the opening of the first shielding member is viewed from an upstream side in the advancing direction of the particle beam.

### BRIEF DESCRIPTION OF THE DRAWINGS

FIG. 1 is a plan-view disposition diagram of a particle beam irradiation apparatus according to one embodiment of the present invention.

FIG. 2 is a schematic configuration diagram illustrating a collimator device.

FIG. 3 is a perspective view illustrating the collimator device.

FIG. 4 is a side view illustrating the collimator device.

FIG. 5 is a perspective view illustrating the collimator device.

FIGS. 6A and 6B are perspective views illustrating the collimator device.

FIG. 7 is a conceptual diagram illustrating how a support member is machined.

FIG. 8 is a conceptual diagram illustrating an inter-shielding member separation distance.

FIG. 9 is a conceptual diagram illustrating the inter-shielding member separation distance.

FIGS. 10A and 10B are diagrams illustrating a collimator according to a comparative example.

FIGS. 11A to 11D are diagrams illustrating a collimator device according to a modification example.

FIGS. 12A and 12B are diagrams illustrating a collimator device according to a modification example.

### DETAILED DESCRIPTION

Here, although the particle beam therapy apparatus described above collimates a particle beam after exit from the irradiation unit, the apparatus does not take any countermeasures against the spread of a particle beam in the transport path that reaches the irradiation unit. Therefore, it has been required to accurately constrict the spread of a particle beam that has exited from an accelerator in a transport path.

In this regard, it is desirable to provide a particle beam irradiation apparatus capable of accurately constricting the spread of a particle beam that has exited from an accelerator in a transport path.

The particle beam irradiation apparatus according to the present invention includes the collimator device having at least the first shielding member and the second shielding member provided in the transport path, shielding the particle beam, and respectively having the openings allowing the particle beam to pass in the advancing direction of the particle beam. The collimator device is capable of constricting the particle beam with the opening of the first shielding member in the transport path between the accelerator and the irradiation unit. Here, the second shielding member is spaced from the first shielding member to the downstream side in the advancing direction of the particle beam. Therefore, among the particle beams that have passed through the opening of the first shielding member, one with a large angle with respect to a base axis can be shielded by the second shielding member at a position spaced downstream in the advancing direction of the particle beam. Further, in a case where the opening of the first shielding member is viewed from the upstream side in the advancing direction of the particle beam, the opening of the second shielding member is visible on the downstream side. Therefore, among the particle beams that have passed through the opening of the first shielding member, one with a small angle with respect to the base axis is capable of passing through the opening of the second shielding member. As described above, the spread of the particle beam that has exited from the accelerator in the transport path can be constricted with high accuracy.

The particle beam irradiation apparatus may further include a degrader provided on the transport path and attenuating the particle beam, in which the collimator device may be provided on a downstream side of the degrader in the advancing direction of the particle beam. In this case, the collimator device is capable of accurately constricting the particle beam spread by the degrader.

The collimator device may have a support member supporting at least the first shielding member and the second shielding member. By supporting the first shielding member and the second shielding member with the same support member, the positional accuracy between the first shielding member and the second shielding member can be improved.

The support member may have a first support surface supporting the first shielding member and a second support surface supporting the second shielding member, and the first support surface and the second support surface may be machined surfaces formed by drilling in the same direction perpendicular to the advancing direction of the particle beam. In this case, the first support surface and the second support surface can be formed using a drill of the same machining apparatus, and thus the positional accuracy of both can be improved. Therefore, the positional accuracy between the first shielding member and the second shielding member supported by these support surfaces can be improved.

The support member may support at least one of the first shielding member and the second shielding member from two directions perpendicular to the advancing direction of the particle beam. In this case, the shielding member can be positioned from two directions, and thus positional accuracy can be improved.

A cooling unit may be connected to the support member. In this case, the first shielding member and the second shielding member can be cooled by the common cooling unit via the support member even if the first shielding member and the second shielding member are not provided with separate cooling units.

According to this collimator device, it is possible to obtain the same actions and effects as those of the particle beam irradiation apparatus described above.

According to the present invention, it is possible to provide a particle beam irradiation apparatus capable of accurately constricting the spread of a particle beam that has exited from an accelerator in a transport path.

Hereinafter, a preferred embodiment of a particle beam irradiation apparatus according to the present invention will be described with reference to the drawings. It should be noted that in the description of the drawings, the same elements are denoted by the same reference numerals with redundant description omitted. In the present embodiment, a case where the particle beam irradiation apparatus is a charged particle beam irradiation apparatus will be described. The particle beam irradiation apparatus is applied to, for example, cancer treatment and is an apparatus that irradiates a tumor (irradiation target) in a patient's body with a particle beam such as a proton beam.

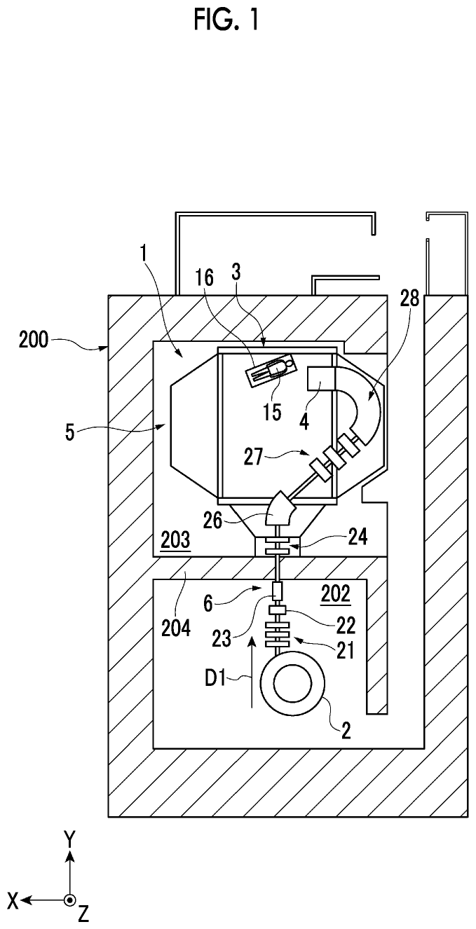

A schematic configuration of the particle beam irradiation apparatus of the present embodiment will be described. FIG. 1 is a plan-view disposition diagram of the particle beam irradiation apparatus according to one embodiment of the present invention. As illustrated in FIG. 1, a particle beam irradiation apparatus 1 includes an accelerator 2 generating a particle beam, an irradiation device 3 having a rotatable irradiation unit 4 irradiating a patient 15 on a treatment table 16 with a particle beam from any direction, and a transport path 6 provided between the accelerator 2 and the irradiation unit 4 and capable of transporting a particle beam. In addition, each device of the particle beam irradiation apparatus 1 is installed in, for example, a room of a building 200. In the present embodiment, the building 200 includes an accelerator room 202 where the accelerator 2 is provided and an irradiation room 203 where the irradiation device 3 is provided.

The irradiation device 3 includes the irradiation unit 4 and a gantry 5. The irradiation unit 4 is a device that irradiates the patient 15 with a particle beam. In a case where the irradiation unit 4 performs irradiation by a scanning method, the irradiation unit 4 is provided with a device such as a scanning electromagnet and another electromagnet. The irradiation unit 4 is attached to the gantry 5 provided so as to surround the treatment table 16. The irradiation unit 4 can be rotated around the treatment table 16 by the gantry 5. The gantry 5 is rotatable around a rotation axis. It should be noted that the irradiation unit 4 has a function of forming a beam into any shape in accordance with the shape of a tumor to be irradiated. The irradiation unit 4 cuts an irradiation field into any shape in the case of a wobbler method and performs beam scanning and beam irradiation at any position in the case of a scanning method. A bending electromagnet 28 of the gantry 5 has a function of changing the direction of a beam and does not form a beam irradiation field. A collimator device may be provided upstream of the irradiation unit 4 in the gantry 5. A degrader is provided upstream of the collimator device and may be provided in the gantry 5.

Here, XYZ coordinates may be set in the following description. The Y-axis direction is the horizontal direction in which the rotation axis of the gantry 5 extends. The X-axis direction is the horizontal direction that is perpendicular to the Y-axis direction. The Z-axis direction is the up-down direction. The rear end side of the gantry 5 is the negative side in the Y-axis direction, and the front end side of the gantry 5 is the positive side in the Y-axis direction. One side in the X-axis direction is a positive side, and the other side is a negative side. The upper side is the positive side in the Z-axis direction, and the lower side is the negative side in the Z-axis direction.

In the present embodiment, the accelerator 2 and the gantry 5 are linearly disposed in the Y-axis direction. Therefore, the transport path 6 extends to the positive side in the Y-axis direction from the accelerator 2 toward the irradiation room 203, passes through a wall portion 204 between the irradiation room 203 and the accelerator room 202, enters the gantry 5 from the rear end side of the gantry 5, and is connected to the irradiation unit 4. In the accelerator room 202, the direction from the negative side toward the positive side in the Y-axis direction is a particle beam advancing direction D1.

A quadrupole electromagnet 21, a degrader 22, a collimator device 23, a quadrupole electromagnet 24, a bending electromagnet 26, a quadrupole electromagnet 27, and the bending electromagnet 28 are provided on the transport path 6 in this order from the accelerator 2 side.

The quadrupole electromagnets 21, 24, and 27 are particle beam-constricting electromagnets. The degrader 22 is a particle beam attenuation member. The collimator device 23 is provided on a downstream side of the degrader 22 in the particle beam advancing direction D1. The collimator device 23 is a device that collimates a particle beam spread by the degrader 22. The bending electromagnet 26 deflects the trajectory of the particle beam transported from the accelerator 2 at the point where the beam enters the gantry 5. The bending electromagnet 28 greatly bends the trajectory of the particle beam such that the particle beam enters the irradiation unit 4 from the outer peripheral side of the gantry 5.

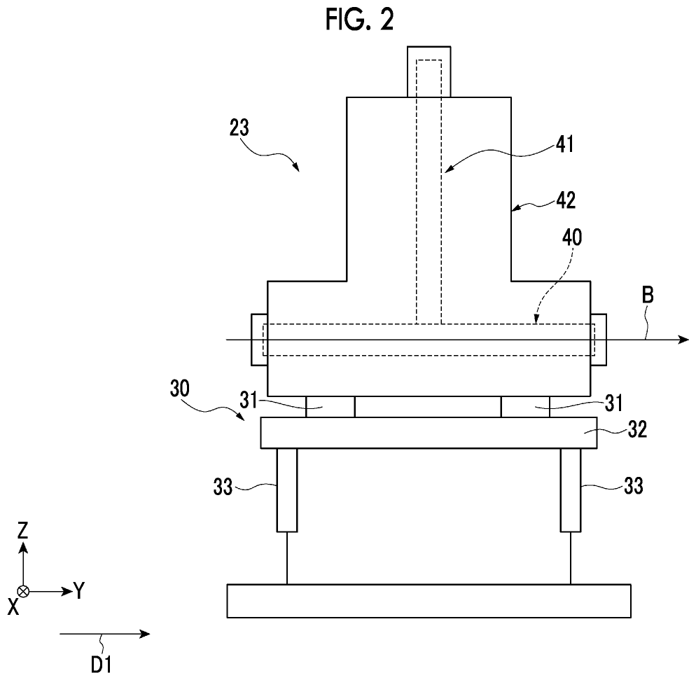

Next, the collimator device 23 will be described with reference to FIG. 2. The collimator device 23 includes a main body 40, a cooling unit 41, and a case 42. The main body 40 is a member that collimates a particle beam B. The main body 40 extends parallel to the Y-axis direction, which is the advancing direction D1. The cooling unit 41 is a mechanism that cools the main body 40. The case 42 accommodates the main body 40 and the cooling unit 41.

The collimator device 23 is provided on a collimator driving device 30. The collimator driving device 30 is a device that adjusts the position and tilt of the collimator device 23. The collimator driving device 30 has an X-axis adjustment mechanism 31 that adjusts the position of the collimator device 23 in the X-axis direction. In addition, the collimator driving device 30 has a Y-axis adjustment mechanism 32 that adjusts the position of the collimator device 23 in the Y-axis direction. In addition, the collimator driving device 30 has tilt adjustment mechanisms 33 that adjust the tilt of the collimator device 23. The tilt adjustment mechanisms 33 are respectively provided on the positive and negative sides in the Z-axis direction and adjust the tilt of the collimator device 23 by performing alignment in the Z-axis direction at the respective positions. Each of the adjustment mechanisms 31, 32, and 33 has a guide member and a drive unit.

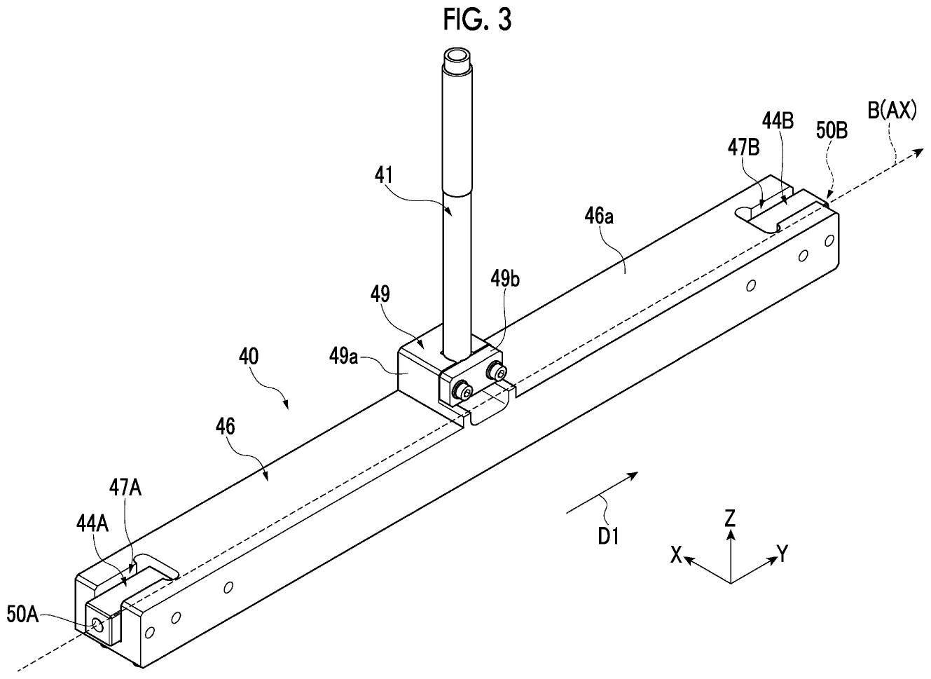

Next, the configuration of the main body 40 of the collimator device 23 will be described in detail with reference to FIGS. 3 to 6A and 6B. As illustrated in FIG. 3, the main body 40 includes a first shielding member 44A, a second shielding member 44B, and a support member 46.

The first shielding member 44A and the second shielding member 44B are members provided in the transport path 6, shielding the particle beam B, and respectively having openings 50A and 50B allowing the particle beam B to pass in the advancing direction D1. The second shielding member 44B is spaced from the first shielding member 44A to the downstream side in the advancing direction D1 (positive side in the Y-axis direction). The shielding members 44A and 44B have a quadrangular prism shape extending along the advancing direction D1. In addition, the openings 50A and 50B are provided at the middle positions of the shielding members 44A and 44B and penetrate the shielding members 44A and 44B in the advancing direction D1.

The shielding members 44A and 44B are made of a material that shields the particle beam B, examples of which include tantalum and tungsten. In addition, the length of the shielding members 44A and 44B in the advancing direction D1 is preferably equal to or greater than the depth of the Bragg peak of the particle beam B. With this length, it is possible to suppress the particle beam B being transmitted through the shielding members 44A and 44B and leaking out at a point other than the openings 50. For example, the 230 MeV proton Bragg peak for tantalum is positioned at a depth of 37.5 mm. Therefore, in a case where tantalum is adopted, the length of the shielding members 44A and 44B in the advancing direction D1 is preferably equal to or greater than 40 mm.

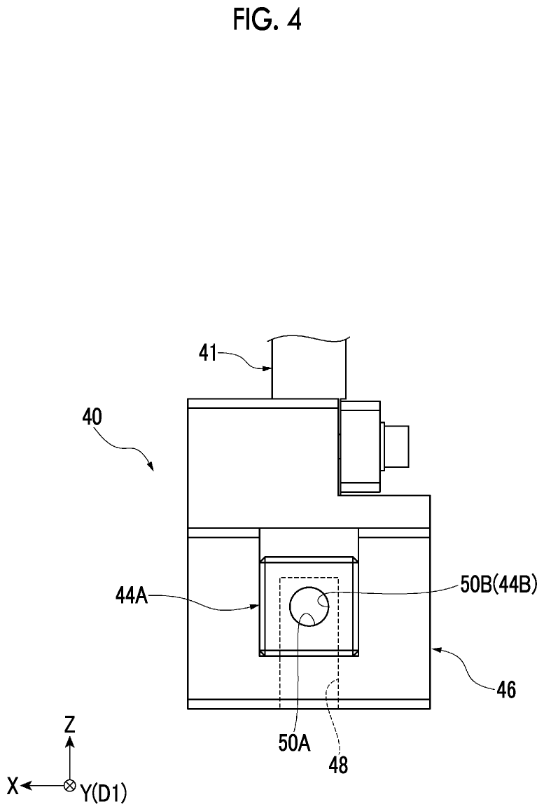

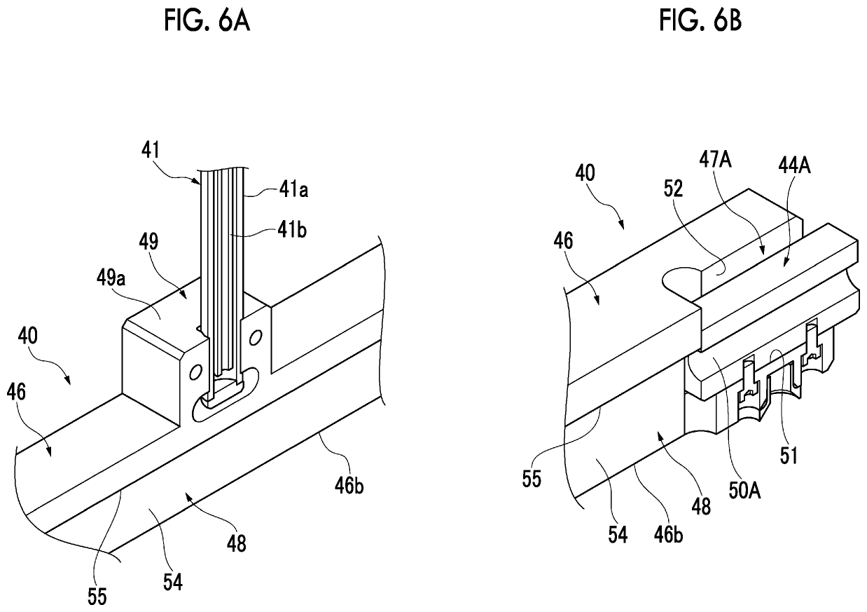

The shielding members 44A and 44B are formed to have the same shape and size and are disposed at the same position on the XZ plane. The opening 50A of the first shielding member 44A and the opening 50B of the second shielding member 44B are columnar through-holes that have the same diameter. The center axis of the opening 50A of the first shielding member 44A and the center axis of the opening 50B of the second shielding member 44B are disposed so as to coincide with a base axis AX of the particle beam B. Here, the support member 46 has a particle beam passage groove portion 48 (see FIGS. 6A and 6B) that forms a passage path for the particle beam B so as not to interfere with the particle beam B. Therefore, in a case where the opening 50A of the first shielding member 44A is viewed from the upstream side in the advancing direction D1 as illustrated in FIG. 4, the opening 50B of the second shielding member 44B is visible on the downstream side. Therefore, the particle beam B incident into the opening 50A of the first shielding member 44A from the upstream side in the advancing direction D1 passes through the particle beam passage groove portion 48 of the support member 46, enters the opening 50B of the second shielding member 44B, and exits from the downstream end portion of the opening 50B in the advancing direction D1.

The support member 46 is a member that supports the first shielding member 44A and the second shielding member 44B. The support member 46 has a quadrangular prism shape extending along the advancing direction D1. The support member 46 is made of a material with high thermal conductivity, examples of which include copper and aluminum.

The support member 46 has a support portion 47A, a support portion 47B, the particle beam passage groove portion 48 (see FIGS. 4, 6A and 6B), and a cooling attachment portion 49.

The support portion 47A is a part for supporting the first shielding member 44A and is formed in the end portion on the upstream side in the advancing direction D1 (negative side in the Y-axis direction). The support portion 47B is a part for supporting the second shielding member 44B and is formed in the end portion on the downstream side in the advancing direction D1 (positive side in the Y-axis direction).

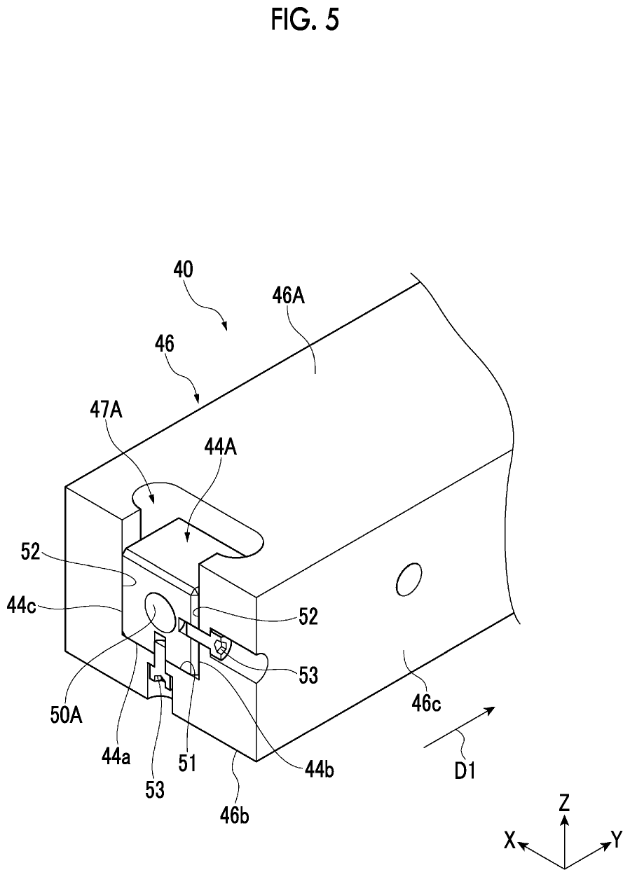

As illustrated in FIG. 5, the support portion 47A is configured as a groove portion that opens upward. The support portion 47A has a support surface 51 (first support surface) parallel to the XY plane and surfaces 52 parallel to the ZY plane. The support surface 51 is provided at a position spaced downward from an upper surface 46a of the support member 46. The support surface 51 supports a lower surface 44a of the first shielding member 44A. The surfaces 52 are provided so as to rise upward from both end portions of the support surface 51 in the X-axis direction. The surface 52 provided with a bolt is “second support surface”.

The surface 52 on the negative side in the X-axis direction supports a side surface 44b of the first shielding member 44A on the negative side in the X-axis direction. The lower surface 44a of the first shielding member 44A is fixed to the support surface 51 by a bolt 53 that is inserted from a lower surface 46b of the support member 46. The side surface 44b of the first shielding member 44A is fixed to the surface 52 by the bolt 53 that is inserted from a side surface 46c of the support member 46 on the negative side in the X-axis direction. With such a configuration, the support member 46 supports the first shielding member 44A from at least two directions perpendicular to the advancing direction D1.

It should be noted that the support portion 47B is configured to be plane-symmetrical with the support portion 47A with respect to the XZ plane at the middle position in the advancing direction D1. Therefore, the support portion 47B has the support surfaces 51 and 52 (second support surfaces) to the same effect as the support portion 47A (see FIG. 6A).

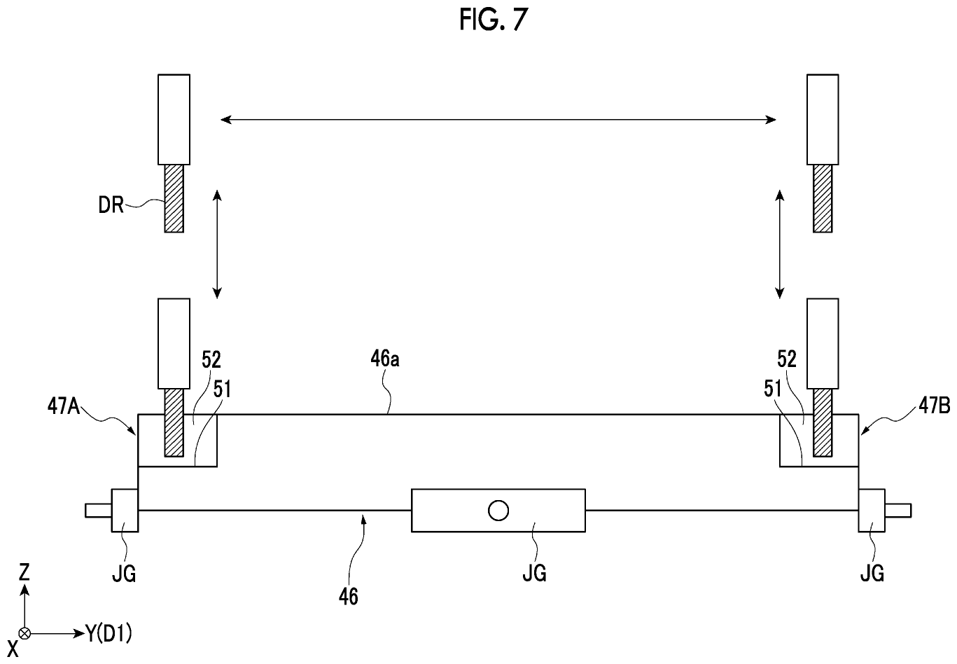

Here, the support surfaces 51 and 52 of the support portion 47A and the support surfaces 51 and 52 of the support portion 47B are machined surfaces formed by drilling in the same direction perpendicular to the advancing direction D1. As illustrated in FIG. 7, the support member 46 that is yet to be machined is fixed with a fixing jig JG. In this state, a drill DR moves in the advancing direction Y in a state of being inserted downward from the upper surface 46a side in the upstream side end portion of the support member 46 in the advancing direction D1, and the support portion 47A is machined as a result. The support surface 51 is formed by machining at the tip part of the drill DR. The support surface 52 is formed by machining at the outer peripheral part of the drill DR.

After the machining of the support portion 47A is completed, the drill DR moves to the downstream end portion of the support member 46 in the advancing direction D1 so as to detour upward so as not to interfere with the support member 46. At this position, the drill DR moves in the advancing direction Y in a state of being inserted downward from the upper surface 46a side, and the support portion 47B is machined as a result. In this manner, the support surfaces 51 and 52 of the support portion 47A and the support surfaces 51 and 52 of the support portion 47B are machined surfaces formed by drilling in the same direction from the positive side to the negative side in the Z-axis direction, which is perpendicular to the advancing direction D1.

When the support portion 47A is machined and when the support portion 47B is machined, the support member 46 remains fixed to the fixing jig JG and does not move. Therefore, once the support member 46 is positioned and fixed, the machining apparatus machines both the support portion 47A and the support portion 47B with one machining program without changing the drill DR. Therefore, the machining apparatus is capable of machining the support portions 47A and 47B with high machining accuracy.

The particle beam passage groove portion 48 is a groove portion that allows the particle beam B to pass and extends along the advancing direction D1 from the support portion 47A to the support portion 47B. As illustrated in FIGS. 6A and 6B, the particle beam passage groove portion 48 is configured as a groove portion that opens downward. The particle beam passage groove portion 48 has a bottom surface 55 parallel to the XY plane and a pair of side surfaces 54 parallel to the ZY plane. The bottom surface 55 is provided at a position spaced upward from the lower surface 46b of the support member 46. The pair of side surfaces 54 are provided so as to extend downward from both end portions of the bottom surface 55 in the X-axis direction. The pair of side surfaces 54 are spaced from each other in the X-axis direction and face each other.

As illustrated in FIG. 3, the cooling attachment portion 49 is provided at the middle position of the upper surface 46a of the support member 46 in the advancing direction D1. The cooling attachment portion 49 has a main body 49a that protrudes upward from the upper surface 46a and a fixing plate 49b that fixes the cooling unit 41 inserted in the main body 49a. As a result, the cooling unit 41 is connected to the support member 46.

As illustrated in FIG. 6A, the cooling unit 41 has an outer tube 41a extending in the up-down direction and an inner tube 41b disposed in the outer tube 41a. The cooling unit 41 causes a cooling medium to flow downward from above through the inner tube 41b. The cooling medium is folded back at the lower end of the inner tube 41b and flows upward from below through the gap between the outer tube 41a and the inner tube 41b. As a result, the support member 46 is cooled via the cooling attachment portion 49.

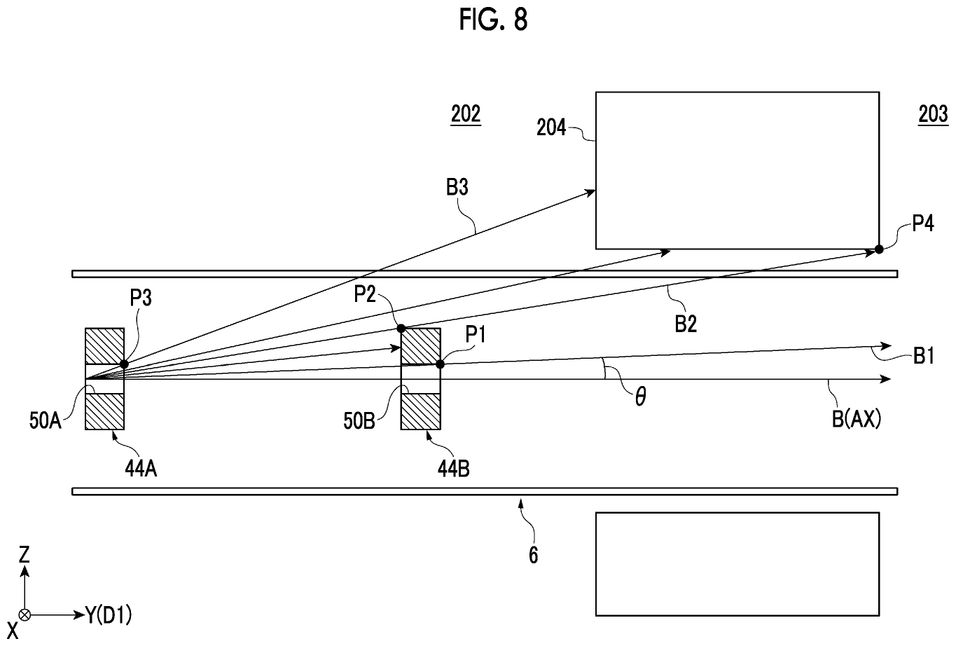

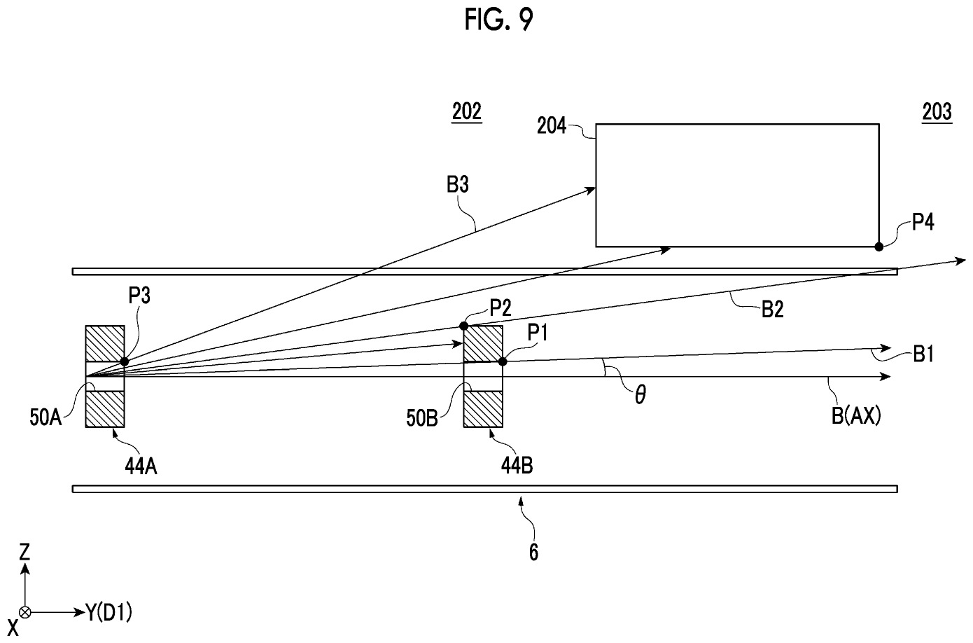

The separation distance in the advancing direction D1 between the first shielding member 44A and the second shielding member 44B will be described with reference to FIGS. 8 and 9. FIGS. 8 and 9 are conceptual diagrams illustrating the state of the transport path 6 near the wall portion 204 separating the accelerator room 202 and the irradiation room 203. In FIGS. 8 and 9, only the shielding members 44A and 44B are illustrated as collimator devices. In addition, an angle θ is the tilt of the component of the particle beam B with respect to the base axis AX. As illustrated in FIG. 8, among the particle beams B that have entered the opening 50A of the first shielding member 44A, the particle beam B with a small angle θ passes through the opening 50B of the second shielding member 44B and is transported to the irradiation room 203 side. Assuming that a particle beam B1 is the particle beam B that passes near an edge portion P1 of the downstream end portion of the opening 50B in the advancing direction D1, one that is smaller in angle θ than the particle beam B1 passes through the opening 50B.

One that is larger in angle θ than the particle beam B1 is shielded by the second shielding member 44B. However, assuming that a particle beam B2 is the particle beam B that passes near an edge portion P2 of the upstream side end portion of the second shielding member 44B in the advancing direction D1, one that is larger in angle θ than the particle beam B2 passes through the outer peripheral side of the second shielding member 44B. Assuming that a particle beam B3 is the particle beam B that passes near an edge portion P3 of the downstream end portion of the opening 50A in the advancing direction D1, one that is smaller in angle θ than the particle beam B3 passes through the opening 50A and the outer peripheral side of the second shielding member 44B. The particle beam B that passes through the outer peripheral side of the second shielding member 44B is prevented from leaking to the irradiation room 203 by the concrete wall portion 204. Leakage into the irradiation room 203 can be prevented if the particle beam B2 hits the wall portion 204 on an upstream side, in the advancing direction D1, of an inner edge portion P4 of the wall portion 204 on the downstream side in the advancing direction D1. As illustrated in FIG. 8, if the separation distance between the shielding members 44A and 44B is kept to a predetermined distance, the particle beam B that is capable of passing through the opening 50B of the second shielding member 44B increases, and thus it is possible to lower the dose of the particle beam B that cannot be used for treatment in the irradiation room 203. In addition, the particle beam B that has passed through the outer peripheral side of the second shielding member 44B can be shielded by the wall portion 204 and prevented from leaking into the irradiation room 203.

In the example of FIG. 9, the separation distance between the shielding members 44A and 44B exceeds that illustrated in FIG. 8. In this case, the angle θ of the boundary particle beam B1 that is capable of passing through the opening 50B decreases. Therefore, the parallelism of the particle beam B used for treatment can be increased. Meanwhile, the boundary particle beam B2 that passes through the outer peripheral side of the second shielding member 44B passes through the wall portion 204 and leaks to the irradiation room 203 side by passing through the inner peripheral side of the inner edge portion P4 of the wall portion 204. As a result, the dose of leaked particle beams that reach the irradiation room 203 and cannot be used for treatment increases.

Next, actions and effects of the particle beam irradiation apparatus 1 and the collimator device 23 according to the present embodiment will be described.

The particle beam irradiation apparatus 1 according to the present embodiment includes the collimator device 23 having at least the first shielding member 44A and the second shielding member 44B provided in the transport path 6, shielding the particle beam B, and respectively having the openings 50A and 50B allowing the particle beam B to pass in the advancing direction D1. The collimator device 23 is capable of constricting the particle beam B with the opening 50A of the first shielding member 44A in the transport path 6 between the accelerator 2 and the irradiation unit 4. Here, the second shielding member 44B is spaced from the first shielding member 44A to the downstream side in the advancing direction D1. Therefore, among the particle beams B that have passed through the opening 50A of the first shielding member 44A, one with a large angle with respect to the base axis AX can be shielded by the second shielding member 44B at a position spaced downstream in the advancing direction D1. Further, in a case where the opening 50A of the first shielding member 44A is viewed from the upstream side in the advancing direction D1, the opening 50B of the second shielding member 44B is visible on the downstream side. Therefore, among the particle beams B that have passed through the opening 50A of the first shielding member 44A, one with a small angle with respect to the base axis AX is capable of passing through the opening 50B of the second shielding member 44B. As described above, the spread of the particle beam B that has exited from the accelerator 2 can be accurately constricted in the transport path 6.

The particle beam irradiation apparatus 1 may further include the degrader 22 that is provided on the transport path 6 and attenuates the particle beam B, and the collimator device 23 may be provided on a downstream side of the degrader 22 in the advancing direction D1. In this case, the collimator device 23 is capable of accurately constricting the particle beam B spread by the degrader 22.

The collimator device 23 may have the support member 46 that supports at least the first shielding member 44A and the second shielding member 44B. By supporting the first shielding member 44A and the second shielding member 44B with the same support member 46, the positional accuracy between the first shielding member 44A and the second shielding member 44B can be improved.

The support member 46 may have the support surfaces 51 and 52 of the support portion 47A supporting the first shielding member 44A and the support surfaces 51 and 52 of the support portion 47B supporting the second shielding member 44B, and the support surfaces 51 and 52 of the support portion 47A and the support surfaces 51 and 52 of the support portion 47B may be machined surfaces formed by drilling in the same direction perpendicular to the advancing direction D1. In this case, the support surfaces 51 and 52 of the support portion 47A and the support surfaces 51 and 52 of the support portion 47B can be formed using the drill DR of the same machining apparatus, and thus the positional accuracy of both can be improved. Therefore, the positional accuracy between the first shielding member 44A and the second shielding member 44B supported by these support surfaces 51 and 52 can be improved.

The support member 46 may support at least one of the first shielding member 44A and the second shielding member 44B from two directions perpendicular to the advancing direction D1. In this case, the shielding members 44A and 44B can be positioned from two directions, and thus positional accuracy can be improved.

The cooling unit 41 may be connected to the support member 46. In this case, the first shielding member 44A and the second shielding member 44B can be cooled by the common cooling unit 41 via the support member 46 even if the first shielding member 44A and the second shielding member 44B are not provided with separate cooling units.

The collimator device 23 according to the present embodiment is the collimator device 23 that collimates the particle beam B, the collimator device 23 includes at least the first shielding member 44A and the second shielding member 44B shielding the particle beam B and respectively having the openings 50A and 50B allowing the particle beam B to pass in the advancing direction D1 and the support member 46 supporting the first shielding member 44A and the second shielding member 44B, the second shielding member 44B is spaced from the first shielding member 44A to the downstream side in the advancing direction D1, and the opening 50B of the second shielding member 44B is visible on the downstream side in a case where the opening 50A of the first shielding member 44A is viewed from the upstream side in the advancing direction D1.

According to this collimator device 23, it is possible to obtain the same actions and effects as those of the particle beam irradiation apparatus 1 described above.

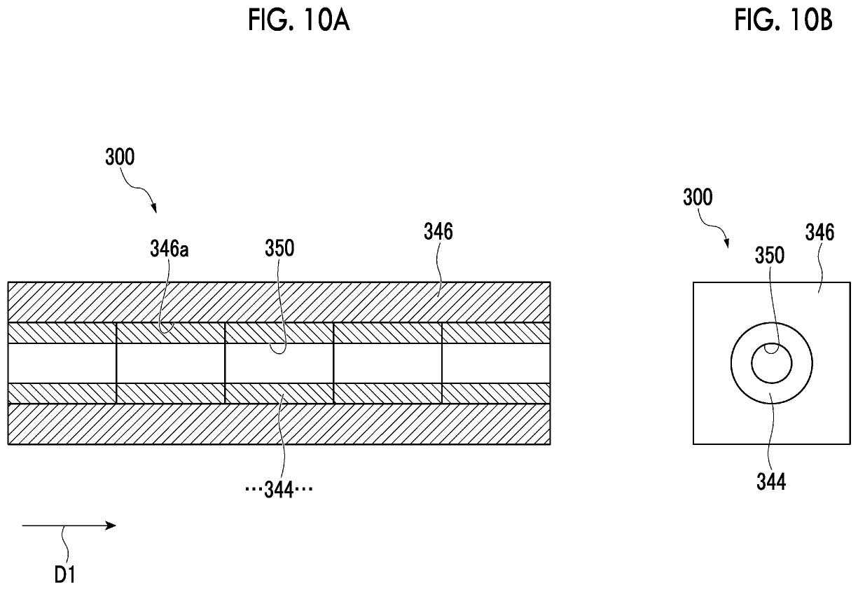

Here, a collimator device 300 according to a comparative example will be described with reference to FIGS. 10A and 10B. The collimator device 300 according to the comparative example has a tubular support member 346 and a plurality of shielding members 344 inserted in the support member 346. The support member 346 has a through-hole 346a that penetrates the support member 346 over the entire length in the advancing direction D1. In addition, the shielding members 344 have openings 350. The shielding members 344 are gaplessly provided such that the openings 350 are connected over the entire length of the support member 346. The collimator device 300 is problematic in that an increase in cost and a decline in dimensional accuracy occur as the number of shielding members 344 increases. On the other hand, in the collimator device 23 according to the present embodiment, it is possible to achieve cost reduction by shielding member omission between the first shielding member 44A and the second shielding member 44B and achieve dimensional accuracy improvement by a decrease in component count. In addition, in order to form the through-hole 346a of the support member 346 in the comparative example, deep hole machining has to be performed with a long drill, and such machining results in a decline in dimensional accuracy and machining limit problems. On the other hand, in the collimator device 23 according to the present embodiment, machining as illustrated in FIG. 7 is possible, and thus machining can be performed with high dimensional accuracy.

The present invention is not limited to the embodiment described above.

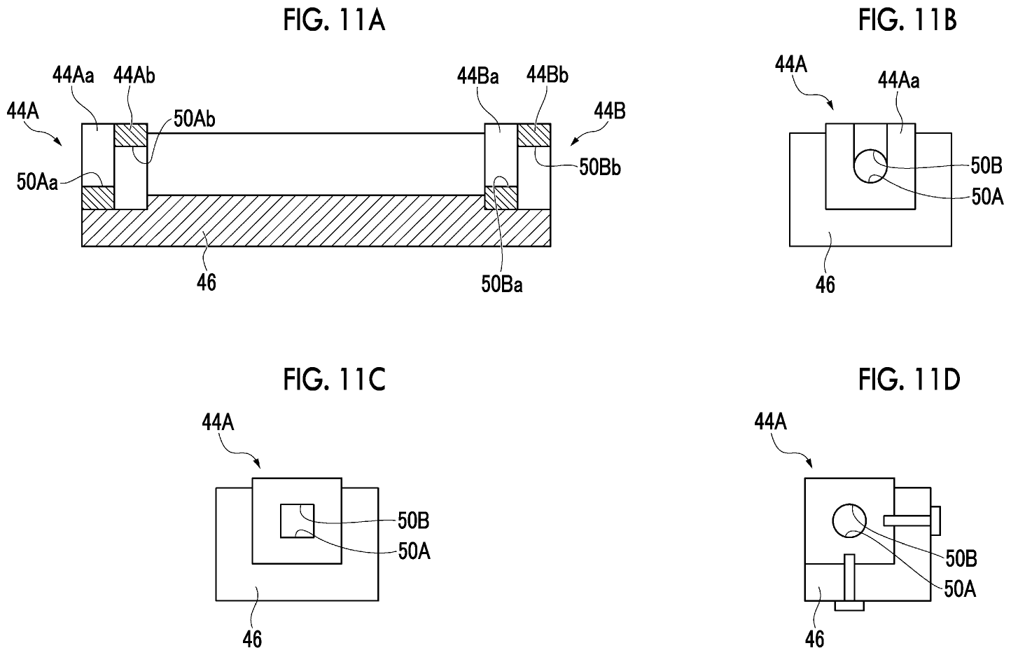

For example, one shielding member may be configured using two pieces as illustrated in FIGS. 11A and 11B. Specifically, as illustrated in FIG. 11A, the first shielding member 44A is configured by combining anti-cracking members 44Aa and 44Ab and the second shielding member 44B is configured by combining anti-cracking members 44Ba and 44Bb. The members 44Aa and 44Ba have U-shaped openings 50Aa and 50Ba. The members 44Ab and 44Bb have inverted U-shaped openings 50Ab and 50Bb. As a result, as illustrated in FIG. 11B, the circular opening 50A is configured by combining the members 44Aa and 44Ab. The circular opening 50B is configured by combining the members 44Ba and 44Bb.

As illustrated in FIG. 11C, the shape of the openings 50A and 50B is not limited and may be a rectangle instead of the circle. In addition, the opening 50A and the opening 50B may differ from each other in shape. In addition, as illustrated in FIG. 11D, the support member 46 may not support the shielding members 44A and 44B with a recessed cross section and may be a structure that supports the shielding members 44A and 44B from two directions with an L-shaped cross section. In this case, the weight of the support member 46 can be reduced.

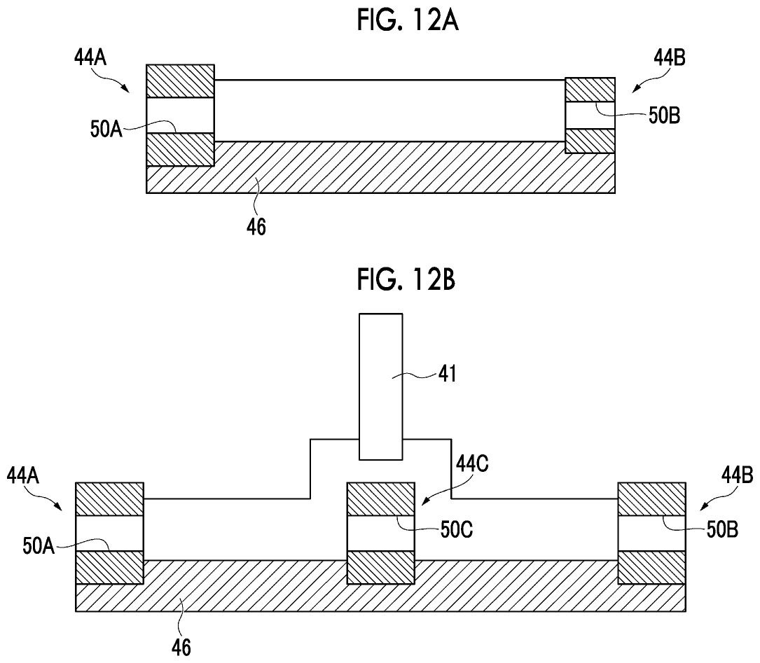

In addition, the second shielding member 44B that is different in size from the first shielding member 44A may be employed as illustrated in FIG. 12A. In this case, it is possible to shield the particle beam B scattered on the upstream side in the advancing direction D1 by increasing the size of the first shielding member 44A. In addition, it is possible to suppress incorrect attachment of the shielding members 44A and 44B during assembly. A third shielding member 44C may be provided as illustrated in FIG. 12B. For example, it is possible to improve the cooling efficiency of the shielding member 44C by providing the shielding member 44C near the cooling unit 41.

For example, the irradiation method of the irradiation unit 4 is not limited to the scanning method described above and, for example, a broad beam method such as wobbler and double scatterer methods may be employed.

The structure of the building 200 and the layout of each component may be changed as appropriate without departing from the gist of the present invention.

It should be understood that the invention is not limited to the above-described embodiment, but may be modified into various forms on the basis of the spirit of the invention. Additionally, the modifications are included in the scope of the invention.

What is claimed is:

1. A particle beam irradiation apparatus comprising:
an accelerator accelerating a particle so as to generate a particle beam;
an irradiation unit irradiating an irradiation target with the particle beam;
a transport path provided between the accelerator and the irradiation unit and provided so as to be capable of transporting the particle beam; and
a collimator device including a first shielding member provided in the transport path, shielding the particle beam, and including a first opening allowing the particle beam to pass in an advancing direction of the particle beam and a second shielding member provided in the transport path, shielding the particle beam, and including a second opening allowing the particle beam to pass in the advancing direction of the particle beam, wherein
the second shielding member is spaced from the first shielding member to a downstream side in the advancing direction of the particle beam, and
the second opening of the second shielding member is visible on the downstream side in the advancing direction of the particle beam in a case where the first opening of the first shielding member is viewed from an upstream side in the advancing direction of the particle beam.

2. The particle beam irradiation apparatus according to claim 1, further comprising a degrader provided on the transport path and attenuating the particle beam,
wherein the collimator device is provided on a downstream side of the degrader in the advancing direction of the particle beam.

3. The particle beam irradiation apparatus according to claim 1, wherein the collimator device includes a support member supporting at least the first shielding member and the second shielding member.

4. The particle beam irradiation apparatus according to claim 3, wherein the support member includes a first support surface supporting the first shielding member and a second support surface supporting the second shielding member, and
the first support surface and the second support surface are machined surfaces formed by drilling in the same direction perpendicular to the advancing direction of the particle beam.

5. The particle beam irradiation apparatus according to claim 3, wherein the support member supports at least one of the first shielding member and the second shielding member from two directions perpendicular to the advancing direction of the particle beam.

6. The particle beam irradiation apparatus according to claim 3, wherein the support member includes a particle beam passage groove portion forming a passage path for the particle beam so as not to interfere with the particle beam.

7. The particle beam irradiation apparatus according to claim 6, wherein the particle beam is incident into the first opening of the first shielding member from the upstream side in the advancing direction of the particle beam, passes through the particle beam passage groove portion of the support member, enters the second opening of the second shielding member, and exits from a downstream end portion of the second opening in the advancing direction of the particle beam.

8. The particle beam irradiation apparatus according to claim 3, wherein a cooling unit is connected to the support member.

9. The particle beam irradiation apparatus according to claim 8, wherein the cooling unit includes an outer tube extending in an up-down direction and an inner tube disposed in the outer tube and causes a cooling medium to flow downward from above through the inner tube.

10. The particle beam irradiation apparatus according to claim 9, wherein the cooling medium is folded back at a lower end of the inner tube and flows upward from below through a gap between the outer tube and the inner tube.

11. A collimator device collimating a particle beam and comprising:
a first shielding member shielding the particle beam and including a first opening allowing the particle beam to pass in an advancing direction of the particle beam and a second shielding member shielding the particle beam and including a second opening allowing the particle beam to pass in the advancing direction of the particle beam; and
a support member supporting the first shielding member and the second shielding member,
wherein the second shielding member is spaced from the first shielding member to a downstream side in the advancing direction of the particle beam, and
the second opening of the second shielding member is visible on the downstream side in the advancing direction of the particle beam in a case where the first opening of the first shielding member is viewed from an upstream side in the advancing direction of the particle beam.
